(19) United States
(12) Patent Application Publication
(10) Pub. No.: US 2023/0132863 A1
(43) Pub. Date: May 4, 2023

(54) MEDICAL SYSTEM FOR TREATING STENOSIS IN INTRACRANIAL VESSELS

(71) Applicant: Acandis GmbH, Pforzheim (DE)

(72) Inventors: Michael Büchert, Pforzheim (DE); Giorgio Cattaneo, Karlsruhe (DE)

(21) Appl. No.: 17/786,476

(22) PCT Filed: Dec. 10, 2020
(86) PCT No.: PCT/EP2020/085399

§ 371 Date: Nov. 16, 2022

(30) Foreign Application Priority Data
Dec. 20, 2019 (DE) 10 2019 135 498.6

(51) Int. Cl. A61F 2/07; A61F 2/958; A61L 31/10

(52) U.S. Cl. CPC A61F 2/07; A61F 2/958; A61L 31/10; A61F 2002/072; A61F 2250/0098; A61F 2210/0076

(57) ABSTRACT

The disclosure relates to a medical system for treating stenosis in intracranial blood vessels including a compressible and self-expandable implant for covering the stenosis, said implant having a lattice structure, at least some sections of which are provided with a cover made of an electrospun fabric, wherein the fabric has irregularly sized pores, and a balloon catheter for dilating the stenosis and/or introducing the implant into the blood vessel.

The invention relates to a medical system with a stent and a balloon catheter for the treatment of stenoses in intracranial vessels.

WO 2014/177634 A1 describes a highly flexible stent which is suitable for supporting blood vessels and has a compressible, and expandable mesh structure, wherein the mesh structure is formed in one piece. The mesh structure comprises closed cells which are each delimited by four mesh elements. The mesh structure has at least one cell ring which comprises between three and six cells.

Furthermore, in the Applicant's experience, stents with mesh structures are known which are formed from a single wire. The wire is braided with itself in order to form a tubular network. At the axial ends of the tubular network, the wire is curved round so that loops which act atraumatically are formed. The axial ends may flare outwards in a funnel shape.

The medical stent is particularly suitable for use in intracranial blood vessels. Blood vessels of this type have a comparatively small cross sectional diameter and are often highly tortuous. For this reason, the known stent is highly flexible in configuration, so that on the one hand it can be compressed to a very small cross sectional diameter, and on the other hand it has a high bending flexibility, which enables it to be delivered through small cerebral blood vessels.

Stenoses are constrictions in blood vessels which lead to a variation in the fluid dynamics of the bloodstream. This can result in a raised blood pressure which leads to stress on the vessel walls, in particular in the region of the stenosis. In the worst case scenario, this can lead to detachment of tissue or tissue rupture. The primary danger is underperfusion because of the constriction or occlusion of the lumen and because of the detachment of particles which then float away distally and occlude smaller distal vessels. Stenoses occur because of changes in the vessel wall due to inflammatory processes (atherosclerotic changes).

In order to regress a stenosis, the use of stents is known. They can be guided to the treatment site via a balloon catheter. Once there, the stenosis is dilated with the aid of the balloon on the balloon catheter. The stent which is expanded in the region of the stenosis then supports the blood vessel. The disadvantage with the use of known stems, however, is that the mesh structure of the stent pushes into the constricted and often already inflamed vessel wall due to the forces generated by dilation and therefore can result in lesions, which in turn can lead to the formation of thrombi. Small thrombi can float away distally and lead to occlusions. In addition, the atherosclerotic tissue itself is fragile, it can rupture in contact with the mesh structure and float away. The meshes of the stent are, on the other hand, so large that thrombi can no longer be safely retained.

In addition, known stents only grow into the tissue with difficulty and therefore form a long-term obstacle for the bloodstream, on which thrombi can be formed.

In the light of the foregoing, the objective of the invention is to provide a medical system for the treatment of stenoses, with a balloon catheter and an implant, with which a treatment site in intracranial blood vessels can easily be reached and with which the side effects of stenosis therapy are reduced.

In accordance with the invention, this objective is achieved by means of the subject matter of patent claim 1.

Thus, the invention is built around the concept of a medical system for the treatment of stenoses in intracranial blood vessels, with a compressible and self-expandable implant for covering the stenosis, which has a mesh structure at least a section of which is provided with a covering produced from an electrospun fabric, and which has irregularly sized pores, as well as with a balloon catheter for dilating the stenosis and for delivering the implant into the blood vessel.

The inventive combination of the implant with the electrospun covering and the balloon catheter has particular advantages in the treatment of stenoses in intracranial blood vessels. The self-expandable implant can be disposed in the region of the stenosis, wherein a close contact with the vessel wall is produced because of the self-expandable properties of the implant. Using the balloon catheter, it is possible to dilate the stenosis before and after placement of the implant. The first dilation is carried out with the balloon of the balloon catheter. Micro-lesions of the blood vessel which occur in this phase can be screened by the covering in the second step of the treatment.

In particular, by means of the balloon, a relatively high expansion pressure may be produced which expands the constricted blood vessel. In the implanted state, the self-expandable implant exerts a permanent radial force on the vessel wall and therefore ensures that it will not bring about a fresh constriction or stenosis. In this manner, because of its fine structure on the one hand, the electrospun covering ensures that the implant connects well with the vessel wall and in particular grows into it, because the electrospun covering supports the formation of endothelial cell tissue. Because of the covering, the force on the vessel is firstly somewhat more homogeneously distributed. In addition, the covering ensures that small thrombi which form on the injured vessel wall cannot float further away distally. The fine-pored covering offers a good substrate for cell proliferation and therefore for the formation of a new endothelial layer.

On the other hand, the covering is highly flexible and thin, so that the implant can be compressed well and has good bending flexibility. Thus, the implant can be correctly introduced by means of the balloon catheter into blood vessels in the intracranial zone which are often narrow, tortuous and small.

The fine-pored electrospun covering also acts to distribute the radial force of the implant as well as the expansion force of the balloon over a relatively large contact surface area, so that “incising” of the mesh structure into the vessel wall tissue is prevented. In this regard, in particular, the electrospun covering also stabilizes what are known as vulnerable plaques (soft plaques) which often form in the region of stenoses. Vulnerable plaques are often a consequence of an arteriosclerotic disease and are also one of the major causes of stenoses. Usually, deposits of fat-rich deposits (plaques) lead to a constriction, i.e. a stenosis, of the blood vessel. Vulnerable plaques can rupture and in the end trigger thrombosis. With the aid of the fine-pored electrospun covering, however, vulnerable plaques are stabilized, so that the risk of rupture and therefore the risk of thrombosis can be reduced.

Particularly preferably, the covering comprises at least 10 pores with a size of between 5 μm² and 15 μm² and/or at least 15 μm² over an area of 100000 μm² . In particular, these pores may be at least 30 μm² in size. The at least 10 pores of the covering may each have an inscribed diameter of at least 4 μm, in particular at least 5 μm, in particular at least 6 μm, in particular at least 7 μm, in particular at least 8 μm, in particular at least 9 μm, in particular at least 10 μm, in particular at least 12 μm, in particular at least 15 μm, in particular at least 20 μm.

During the course of manufacture of the covering, the minimum size of the pores can in particular be adjusted by the process time for electrospinning. Moreover, the covering produced from an electrospun fabric is thin and flexible, which promotes the flexibility of the mesh structure. In particular, in contrast to prior art coverings which are produced from other textile materials, the covering of the mesh structure barely affects the compressibility. Overall, then, the implant can be compressed to a considerably smaller cross sectional diameter and therefore can readily be introduced into particularly small blood vessels.

The good flexibility of the support structure or the mesh structure may in particular be achieved when the mesh structure has a closed cell ring which has at most 12 immediately adjacent cells in the circumferential direction of the mesh structure. The closed cell ring also means that after partial release, the mesh structure can be pulled back into a balloon catheter 60, since no mesh elements which could become stuck on the tip of the catheter protrude out because of the closed structure. In particular, all of the cell rings of the mesh structure may be closed and have at most 12, in particular at most 10, in particular at most 8, in particular at most 6 immediately adjacent cells in the circumferential direction of the mesh structure. It is possible for all of the cell rings to comprise at least 3 cells which are immediately adjacent in a circumferential direction of the mesh structure.

By limiting the cells in the circumferential direction to a cell ring, the mesh elements as well as their connectors or points of intersection are also limited. Because of the limited number of mesh elements in the circumferential direction, the mesh structure can be compressed to a small cross sectional diameter in which the mesh elements preferably lie immediately adjacent to each other. Moreover, by limiting the cells in the circumferential direction, a higher bending flexibility can also be obtained, so that the mesh structure, in particular even in the compressed state, can be guided through narrow tortuous vessels by means of the balloon catheter.

Preferably, the mesh elements delimit closed cells of the mesh structure, wherein each dosed cell is delimited by four respective mesh elements. The closed cells may have a diamond-shaped basic structure. The closed cells mean that the mesh structure has high stability, which is advantageous having regard to the function of the mesh structure as a support for the covering. In particular, a high stability is obtained in the axial direction, i.e. in the direction of a longitudinal axis of the mesh structure, which improves delivery of the implant through the balloon catheter. In the radial direction, because of the closed cells, the mesh structure may have an enhanced flexibility, which results in an improved radial force.

In general, in the context of the present invention, all of the geometrical information regarding the implant are with reference to the neutral state of the implant. The neutral state indicates the expanded state of the implant, in which the implant exerts no radial forces.

The balloon catheter may have at least two channels and a balloon, wherein an inflation channel is in fluid communication with the balloon, and a delivery channel extends through the balloon. The delivery channel may have a proximal inlet opening and a distal outlet opening for deploying the implant. Of the at least two channels, the inflation channel is provided for filling the balloon with a fluid in order to dilate the balloon or to discharge the fluid from the balloon in order to compress the balloon. The fluid may be a sodium chloride solution, in particular with a contrast agent for visualization under radiographic monitoring.

Preferably, the delivery channel is configured as a through channel and enables the implant to be guided to the treatment site. Because the implant has good compressibility because of the thin and flexible covering, the delivery channel can be correspondingly small. In addition, the particular structure of the electrospun covering means that the implant can be pushed through the delivery channel with a comparatively low frictional resistance. In this regard, in a preferred variation of the medical system in accordance with the invention, in the compressed state, the implant can be moved through the delivery channel or is disposed in the delivery channel.

In order to further facilitate delivery of the implant, the delivery channel may furthermore have a friction-reducing inner coating for a translational movement of the implant in the delivery channel.

In particular, the suitability of the medical system for use in intracranial blood vessels is promoted when the delivery channel has an internal diameter of at most 991 μm (0.039 inches), in particular at most 686 μm (0.027 inches), in particular at most 635 μm (0.025 inches), in particular at most 533 μm (0.021 inches), in particular at most 432 μm (0.017 inches), in particular at most 406 μm (0.016 inches), in particular at most 381 μm (0.015 inches), in particular at most 330 μm (0.013 inches).

The balloon catheter may have at least one X-ray marker. Preferably, the balloon catheter 60 comprises three X-ray markers, wherein a first X-ray marker is disposed in the region of the distal outlet opening of the delivery channel, a second X-ray marker is disposed in the region of a distal balloon end and a third X-ray marker is disposed in the region of a proximal balloon end. The X-ray marker enables an operator to detect and if necessary correct the position of the balloon inside a blood vessel under radiographic monitoring.

In order to ensure that the covering enables correct deposition of endothelial cells on the one hand and a :large contact surface area with the vessel wall on the other hand, preferably, the pores are at most 750 μm² in size, in particular at most 500 μm² in size, in particular at most 300 μm² in size. For good stenosis treatment and for good covering of the plaques, it has in addition been shown to be appropriate for at, most 20% of the area of the covering to be formed by pores at least 500 μm² in size. As an alternative or in addition, at most 50% of the surface area of the covering may be formed by pores which are at least 300 μm² in size.

The covering may be securely connected to the mesh structure, in particular mechanically interlocked. In particular, the covering may be applied directly to the mesh structure. As an example, the process of electrospinning may be carried out directly on the mesh structure, so that with the formation of the covering, at the same time a connection with the mesh structure is produced. The covering may be mechanically interlocked with the mesh structure. As an example, the covering may be connected to the mesh structure by means of an adhesive bond. The adhesive bond may be produced using a bonding agent. As an example, the bonding agent may comprise or consist of polyurethane.

The secure connection between the covering and the mesh structure prevents detachment of the covering from the mesh structure when the implant is delivered through the delivery channel of the balloon catheter. At the same time, positioning of the implant is facilitated under radiographic monitoring, because it, is sufficient to apply appropriate X-ray markers either to the mesh structure or to the covering. Because the relative positioning between the covering and mesh structure remains the same, additional implant X-ray markers which would make a relative displacement between the covering and mesh structure detectable are not necessary. In total, the number of implant X-ray markers, for example X-ray marker sheaths, can therefore be reduced, which in turn has a positive effect on the compressibility of the implant.

The mesh structure may be sheathed with a bonding agent, in particular polyurethane, at least in parts and/or in sections. In particular, the bonding agent may form the mechanical interlock between the covering and the mesh structure. Preferably, the bonding agent surrounds the entire mesh element and in this manner forms a sheath for the mesh element. Specifically, the mesh elements of the mesh structure may be sheathed with a bonding agent, in particular polyurethane. In all cases, the bonding agent can form the mechanical interlocking connection between the covering and the mesh structure. The bonding agent advantageously surrounds the entire mesh element and in this manner forms a sheath for the mesh element.

In a preferred embodiment of the invention, the covering is disposed on an outside and/or on an inside of the mesh structure. In this situation, the mesh structure forms a support structure which applies a sufficient radial force in order to fix the covering against a vessel wall. In this regard, the mesh structure supports the externally disposed covering. As an alternative, the covering may also be disposed on an inside of the mesh structure.

As an alternative or in addition, the covering may be disposed on an inside of the mesh structure. In particular, it is possible for the mesh structure to be incorporated between two coverings which are respectively formed by an electrospun fabric. In this manner, the mesh elements of the mesh structure may be completely sheathed by the electrospun fabric. Specifically, it may be provided that the electrospun fabric of a covering extends on the inside of the mesh structure in through the cells of the mesh structure and is connected to the electrospun fabric of a covering on the outside of the mesh structure. The mesh elements which border the cells are therefore sheathed on all sides by electrospun fabric.

Preferably, the covering is formed by a plastic material, in particular a polyurethane. Materials of this type have high ductility and can readily be produced in fine filaments by means of an electrospinning process. On the one hand, the plastic material can therefore be used to produce a particularly thin and fine-pored covering. On the other hand, the plastic material already has a high intrinsic flexibility, so that a high compressibility of the implant is obtained.

When using polyurethane in order to form the covering, it has been shown to be particularly advantageous when the Shore hardness of the polyurethane is more than 60 D, in particular at least 65 D, in particular at least 75 D. A particularly preferred range is 65 D-75 D.

Preferably, the same material is used to form the bonding agent and to form the covering. The layer thickness of the bonding agent is preferably between 0.1 μm and 3 μm, in particular between 0.2 μm and 2 μm, in particular between 0.3 μm and 1 μm. The layer thickness of the covering is preferably between 1 μm and 35 μm, in particular between 2 μm and 25 μm, in particular between 3 μm and 15 μm, in particular between 5 μm and 10 μm.

A contribution to the flexibility of the covering is made when, as is preferable, the covering is formed from irregular filaments disposed in a network-like manner which have a filament thickness of between 0.1 μm and 3 μm, in particular between 0.2 μm and 2 μm, in particular between 0.3 μm and 1.5 μm, in particular between 0.7 μm and 1.3 μm.

The covering may have a biocompatible coating. In particular, the coating may be anti-inflammatory and/or hyperplasia-inhibiting. It is also possible for the coating to have antithrombogenic and/or endothelialisation-promoting properties. The coating may contain fibrin and/or heparin. Preferably, the heparin is covalently bonded to the fibrin or incorporated into fibrin.

In particular as a supplement to the mechanically stabilizing effect of the electrospun covering in the case of vulnerable plaques, the heparin contributes to reducing the risk of thrombus. The anti-inflammatory coating, primarily in the combination of fibrin and heparin, preferably covalently bonded, also contributes to regression of vulnerable plaques.

A fibrin coating may consist of a fibrin nanostructure (fibrin filaments); these fibrin filaments form a random network on the surface of the covering and provide an additional surface with which an anticoagulant can be bonded. The fibrin coating which is formed on the network structure may contain an anticoagulant which comprises heparin or other possible functional molecules such as fibronectin, for example.

In a preferred embodiment of the invention, the covering of the mesh structure and/or the balloon of the balloon catheter comprises, in particular contains, a pharmaceutically effective substance or is coated therewith. In particular, a pharmaceutically effective substance may be incorporated into the material of the covering. In general, the pharmaceutically effective substance may be a substance which is released at the vessel wall of the blood vessel. The substance is released from the balloon and then in the second step is held at the relevant site by the covering.

In general, the mesh structure may be configured as a single-pieced mesh structure. It is also possible for the mesh structure to be formed from mutually braided wires. In this regard, in preferred embodiments, the mesh elements form webs which are coupled together into one piece by means of web connectors (one-piece mesh structure). As an alternative, the mesh elements may form wires which are braided with each other (braided mesh structure). While a braided mesh structure is characterized by a particularly high flexibility, in particular bending flexibility, a one-piece mesh structure has comparatively thin walls, so that the mesh structure has a smaller influence on the blood flow inside a blood vessel.

The covering may have a ductility in accordance with ASTM 412 of between 300% and 550%, in particular between 350% and 500%, in particular between 375% and 450%. The elastic modulus of the covering in accordance with ASTM 412 may be:

at 50% extension: >15-21 MPa (psi)
at 100% extension: >18<26 MPa (psi)
at 300% extension: >32<41 MPa (psi),

The Shore hardness of the covering in accordance with ASTM D 2240 may be between 80 A and 85 D, in particular between 90 A and 80 D, in particular between 55 D and 75 D.

In order to improve the ability to be repositioned, after compression and renewed deployment of the mesh structure, the covering may be capable of returning to its original configuration, in particular its non-folded configuration.

The filaments or monofilaments of the fabric may be securely connected to each other at their points of intersection in the fabric in order to prevent them from slipping over each other. This ensures the porosity/pore size which is established by the production process. The cohesive connection is also provided after compression, delivery through the catheter and renewed deployment of the implant in the vessel and persists even when a side branch is perfused through the fabric.

In addition to the pores formed by electrospinning, the fabric may also be perforated by further pores which are formed in the electrospun fabric by processing the fabric, in particular by laser cutting. In this manner, a deliberate and, if desired, regional increase of the porosity or increase in pore size is obtained after the electrospinning process. As an example, laser cut, defined pores may be formed over the entire circumference or additionally over only a portion thereof.

The fabric is preferably perforated by the further pores over at least 25%, in particular over at least 40%, in particular over at least 50% of the circumference of the mesh structure (10). In this regard, for example, the region opposite the neck of the aneurysm can be deliberately perforated.

At least 25%, in particular at least 40%, in particular at least 50% of the circumference of the mesh structure may be free from further pores. In other words, a portion of the fabric is not post-processed or subsequently perforated. In this portion of the fabric, no further pores in addition to the pores formed by electrospinning are introduced into the fabric. In this region, the fabric consists only of the pores formed by electrospinning. The region of the fabric which is free from further pores may be disposed in the region of the neck of the aneurysm when in the implanted state. This may be desired, for example, when an unchanged porosity of the electrospun fabric is advantageous to the treatment of the aneurysm.

A combination of regions of unaltered electrospun fabric and subsequently perforated electrospun fabric is possible.

Starting from the axial centre of the mesh structure, the further pores may be formed in both axial directions. In a further exemplary embodiment, additional pores may be disposed proximally or distally within the cover or the fabric.

The length over which the further pores may be distributed corresponds to at least 25% of the axial length of the covering or of the fabric, in particular at least 30%, in particular at least 40%, in particular at least 50% of the axial length of the covering or of the fabric.

In order to promote perfusion, the size of the further pores may be at least 50 μm, in particular at least 100 μm, in particular at least 200 μm, in particular at least 300 μm.

The separation of the further pores with respect to each other may be at least 1 multiple, in particular at least 1.5 multiples, in particular at least 2 multiples, in particular at least 2.5 multiples of the diameter of the further pores. The term “1 multiple” here means the diameter of a further pore.

In a particularly preferred embodiment, the circumferential contour of the covering is marked by a radiopaque means, at least in sections, in particular entirely circumferentially. This may, for example, be obtained by using radiopaque wires which are braided into the mesh structure along the contour of the covering. It is also possible to obtain the contour of the covering by means of an array of radiopaque sheaths, for example Pt—Ir sheaths or crimped C sheaths.

The site of the covering or fabric is therefore visible under X rays, so that the physician can securely position the device—even in the correct rotational position.

The fabric per se may have a radiopaque means. As an example, the filaments of the fabric may be filled with a radiopaque material, in particular with at least 10% to a maximum of 25% of radiopaque material, for example barium sulphate, BaSO₄. The base colour of the filaments of the fabric may be transparent; upon adding barium sulphate, BaSO₄, these can appear white/yellowish.

The invention will now be explained in more detail with the aid of exemplary embodiments and with reference to the accompanying drawings, in which:

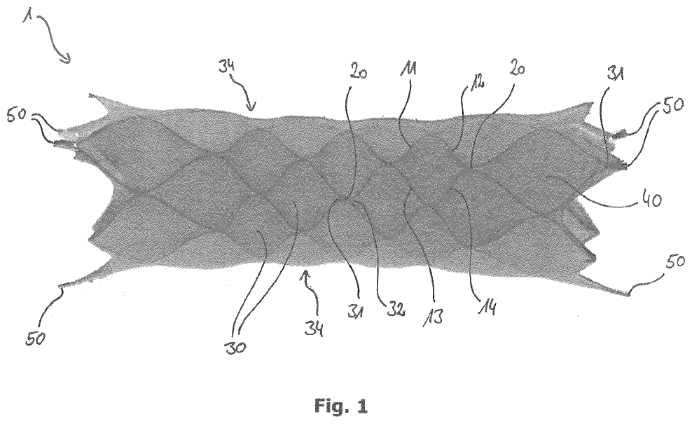

FIG. 1: shows a side view of a stent of a medical system in accordance with the invention according to a preferred exemplary embodiment;

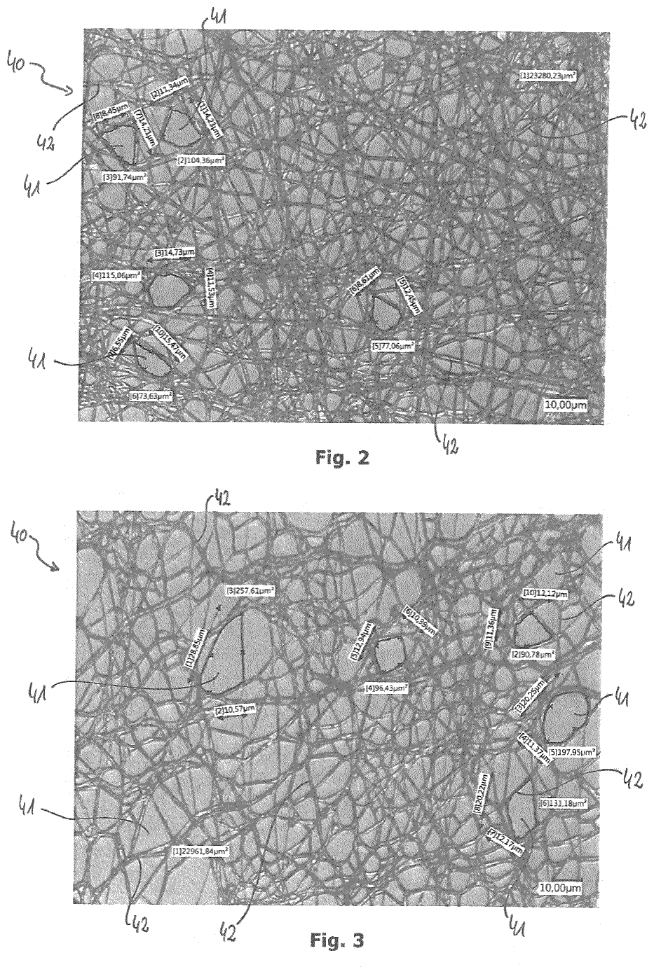

FIG. 2: shows a microscope image of a covering of the implant of a medical system in accordance with the invention according to a preferred exemplary embodiment;

FIG. 3: shows a microscope image of a covering of the implant of a medical system in accordance with the invention according to a further exemplary embodiment;

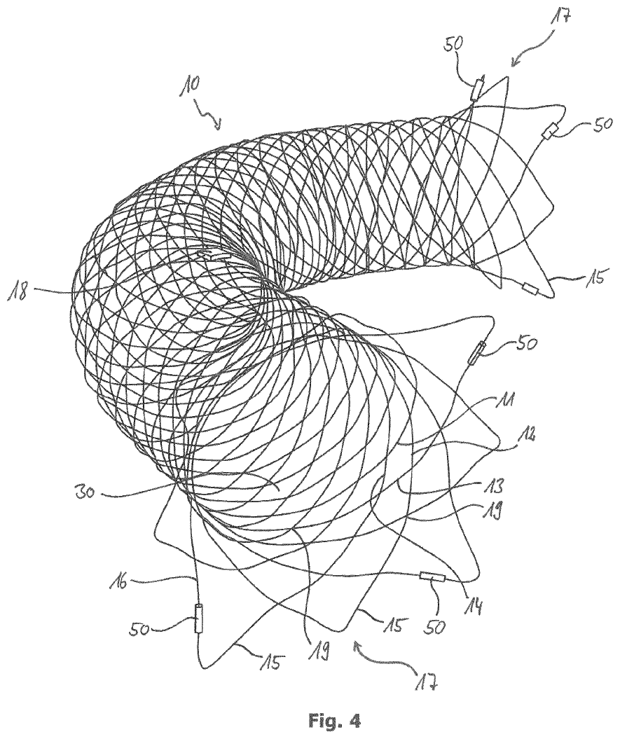

FIG. 4: shows a perspective view of a mesh structure of a stent of a medical system in accordance with the invention according to a further preferred exemplary embodiment;

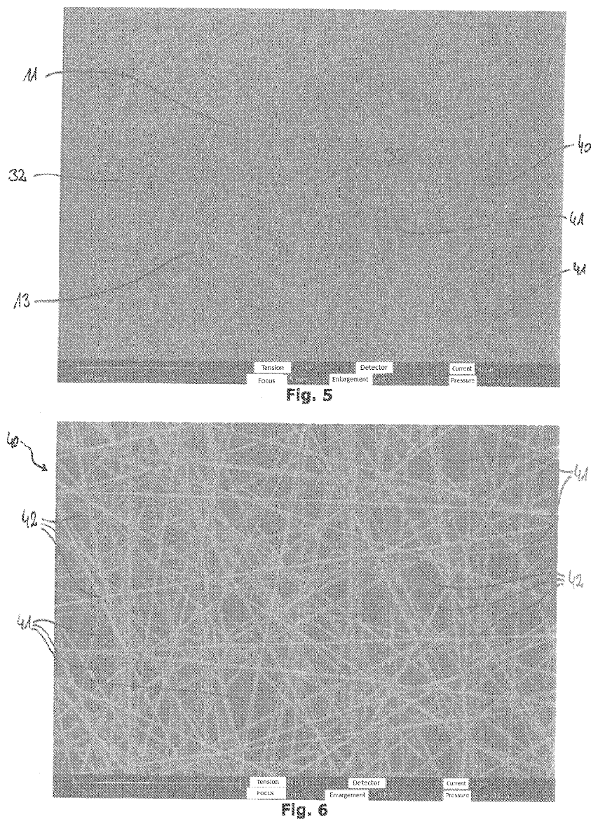

FIG. 5: shows a scanning electron microscope image of a covering of the implant of a medical system in accordance with the invention according to a further preferred exemplary embodiment, at 500× magnification;

FIG. 6: shows a scanning electron microscope image of the covering of FIG. 5, under 3500× magnification;

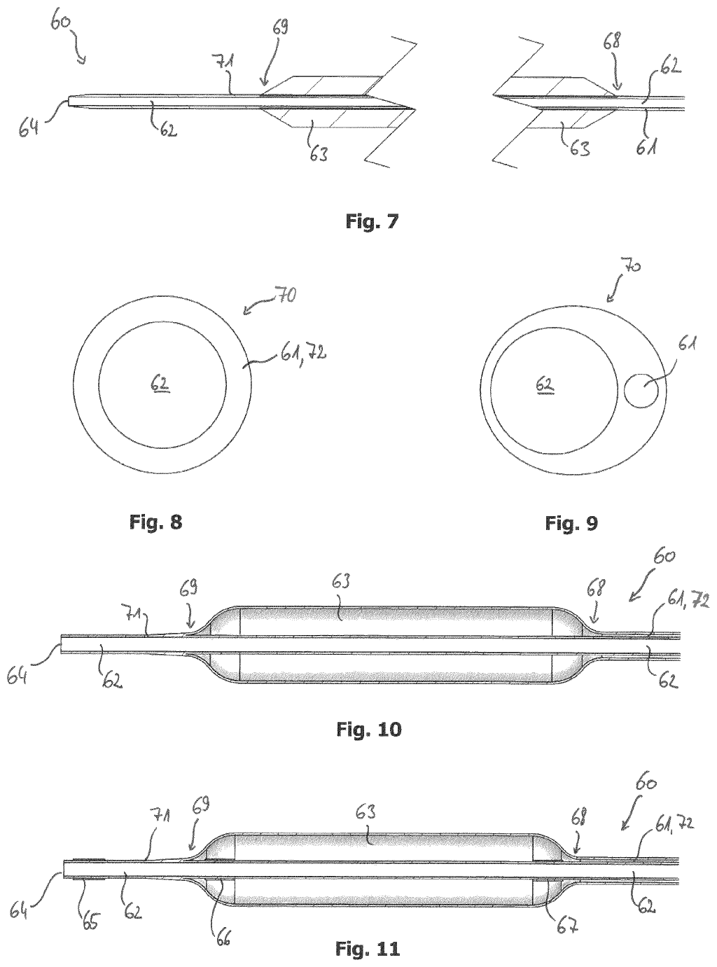

FIG. 7: shows a longitudinal section through the balloon catheter of a medical system in accordance with the invention according to a preferred exemplary embodiment;

FIG. 8: shows a cross section through the balloon catheter of a medical system in accordance with the invention according to a further preferred exemplary embodiment with coaxially disposed channels;

FIG. 9: shows a cross section through the balloon catheter of a medical system in accordance with the invention according to a further preferred exemplary embodiment with adjacent disposed channels;

FIG. 10: shows a longitudinal section through the balloon catheter in accordance with FIG. 8; and

FIG. 11: shows a longitudinal section through the balloon catheter in accordance with FIG. 8, wherein in addition, X-ray markers are provided.

The accompanying figures show an implant in the form of a stent 1 and the balloon catheter 60 of a medical system for the treatment of stenoses in intracranial blood vessels. In particular, the stent 1 has a mesh structure 10 which is compressible and expandable. In other words, the mesh structure 10 may take up a delivery state in which the mesh structure 10 has a relatively small cross sectional diameter. The mesh structure 10 is self-expandable, so that the mesh structure 10 can expand by itself to a maximum cross sectional diameter without the influence of external forces. The state in which the mesh structure 10 has the maximum cross sectional diameter corresponds to the neutral state. In this state, the mesh structure 10 does not exert any radial forces.

Preferably, the mesh structure 10 is one-piece in configuration. In particular, at least sections of the mesh structure 10 may be cylindrical in shape. Preferably, the mesh structure 10 is produced from a tubular blank by laser cutting. In this regard, individual mesh elements or webs 11, 12, 13, 14 of the mesh structure 10 are exposed by the laser cutting process. The regions removed from the blank form cells 30 of the mesh structure 10.

The cells 30 have a substantially diamond-shaped basic shape. In particular, the cells 30 are delimited by four respective webs 11, 12, 13, 14. The webs 11, 12, 13, 14 in the exemplary embodiment that is depicted here have an at least partially curved profile, in particular S-shaped. Other shapes for the webs 11, 12, 13, 14 are possible.

As an example, it is possible for the mesh structure to comprise circumferential segments produced from closed cells, wherein the cells are each delimited by four webs which are coupled together at connection points and of which respectively two are adjacent in the circumferential direction UR of the mesh structure and webs coupled together at a connection point have different flexibilities in a manner such that the web with the higher flexibility is more deformable than the web with the lower flexibility upon the transition of the mesh structure from the expanded state into the compressed state, and of which the webs with higher flexibility and the webs with lower flexibility are respectively diagonally opposite in a manner such that two connection points of the cells which are opposite in the longitudinal direction LR of the mesh structure are displaced against one another in the circumferential direction UR during the transition of the mesh structure from the expanded state into the compressed state. In particular, all of the cells of a circumferential segment are identical in configuration in a manner such that the entire mesh structure twists at least in sections during the transition from the expanded state into the compressed state.

In a further embodiment, the mesh structure may have webs which are connected together into one piece by means of web connectors and delimit closed cells of the mesh structure. The web connectors each have a connector axis which extends between two adjacent cells in the longitudinal direction of the mesh structure. During the transition of the mesh structure from the manufactured state into a compressed state, the web connectors rotate so that an angle between the connector axis and a longitudinal axis of the mesh structure changes upon the transition of the mesh structure from a completely expanded manufactured state into a partially expanded intermediate state, in particular increases. The mesh structure may be configured in one piece. The webs of the mesh structure may, for example, be cut free by laser cutting processing of a tubular blank. The regions which have been cut out from the cells which are delimited by the webs. This is preferably a mesh structure with a closed cell design. The cells are therefore completely surrounded by webs. In particular, the cells may have a substantially diamond-shaped basic shape. In other words, the respective cells are preferably delimited by four webs.

The web connectors which form a one-piece part of the mesh structure can therefore couple four webs together. The web connectors essentially form points of intersection of the webs.

During compression or expansion of the mesh structure, the height and the width of the individual cells of the mesh structure change. The degree of change of the height and width of the cells is influenced by the rotation of the web connectors. In particular, the rotation of the web connectors causes a varying, in particular dynamically varying relationship between the cell height and cell width. This leads to a comparatively high flexibility of the mesh structure, in particular in the direction transverse to the axis. In particular, the rotation of the web connectors enables the mesh structure to become oval when guided through narrow hollow organs of the body. The mesh structure, which may have a cylindrical cross section at least in sections, can therefore take up an oval cross sectional geometry at least locally when being passed through a contorted vessel.

The cells 30 each have cell tips 31, 32 which form the corner points of the diamond-shaped basic shape. The cell tips 31, 32 are respectively disposed at web connectors 20 which each connect four webs 11, 12, 13, 14 together into one piece. Four respective webs 11, 12, 13, 14 extend from each web connector 20, whereupon two cells 30 are associated with each web 11, 12, 13, 14. The webs 11, 12, 13, 14 respectively delimit the cell 30.

FIG. 1 shows the mesh structure 10 in the neutral state. It can readily be seen that the web connectors 20 are substantially respectively disposed on a common circumferential line. Overall, then, a plurality of cells 30 form a cell ring 34 in the circumferential direction of the mesh structure 10. A plurality of cell rings 34 connected together in the longitudinal direction form the entire mesh structure 10. In the exemplary embodiment shown, the cell rings 34 each comprise six cells 30. In this regard, it should be noted here that the mesh structure 10 may advantageously be formed by interconnected cell rings which have the same cross sectional diameter.

When the mesh structure 10 is deployed from the balloon catheter 60, the mesh structure 10 automatically expands radially outwardly. In this regard, the mesh structure 10 passes through a plurality of levels of expansion until the mesh structure 10 reaches the implanted state. In the implanted state, the mesh structure 10 preferably has a cross sectional diameter which is approximately 10% to 30%, in particular approximately 20% smaller than the cross sectional diameter of the mesh structure 10 in the neutral state. Thus, in the implanted state, the mesh structure 10 preferably exerts a radial three on the surrounding vessel walls. The implanted state is also described as the “intended use configuration”.

As can readily be seen in FIG. 1, radiographic implant markers 50 are provided on the stent 1. The radiographic implant markers 50 are disposed at cell tips 31, 32 on the edge cells 30 of the mesh structure 10. Specifically, the radiographic implant markers 50 may be formed as radiopaque sheaths, for example produced from platinum or gold, which are crimped onto the cell tips 31, 32 of the edge cells 30. In FIG. 1, it can be seen that each longitudinal end of the mesh structure 10 has three respective radiographic implant markers 50.

The mesh structure 10 of FIG. 1 can be divided into three sections. Two edge sections, which are each formed by two cell rings 34, are connected via a central section which comprises five cell rings 34. The cells 30 of the central section essentially have a diamond-shaped geometry, wherein all of the webs 11, 12, 13, 14 of the cells 30 of the central section have substantially the same length. The edge cell rings 34 each comprise cells 30 in which two of the immediately adjacent webs 11, 12, 13, 14 in the circumferential direction are each longer in configuration than the two webs 11, 12, 13, 14 of the same cell 30 which are adjacent in the axial direction. In this manner, the edge cells 30 essentially form a kite-like basic shape.

The medical device of FIG. 1 furthermore comprises a covering 40 which is disposed on an outside of the mesh structure 10. The covering 40 bridges the entire mesh structure 10 and in particular covers the cells 30. The covering 40 is produced from an electrospun fabric and is therefore characterized by a particularly thin wall. At the same time, the covering 40 is sufficiently stable to follow an expansion of the mesh structure 10. Preferably, the covering 40 is completely and securely connected to the mesh structure 10. Specifically, the covering 40 is preferably bonded to the webs 11, 12, 13, 14, for example by means of a bonding agent.

The covering 40 may extend over the entire mesh structure 10, as can be seen in FIG. 1. Alternatively, it is possible for the covering 40 to extend over only a portion of the mesh structure 10. As an example, edge cells 30 at one axial end or at both axial ends of the mesh structure 10 may be without a covering. In this regard, the covering 40 may stop before the last or penultimate cell ring 34 of the mesh structure 10. The cell rings 34 which are without a covering allow for good coupling to a transport wire. The region of the stent which has the covering 40 can be highlighted by further radiographic implant markers. It is also possible for radiopaque material to be incorporated into the covering.

The construction of the covering 40 can readily be discerned from the microscope images of FIGS. 2 and 3. These show that the covering 40 has a plurality of irregularly sized pores 41 which are each delimited by filaments 42. By means of the electrospinning process, a plurality of filaments 42 are formed which are orientated in an irregular manner with respect to each other. The pores 41 are formed in this manner. FIG. 2 also shows that the pores 41 have comparatively small pore sizes, wherein some pores 41 are sufficiently large, however, to ensure blood permeability. Specifically, in FIG. 2, four pores 41 with a size of more than 30 μm² have been graphically highlighted. The density of the pores 41 with a size of more than 30 μm² indicates that the covering has at least 10 pores 41 of this type over an area of 100000 μm².

FIG. 3 shows a further exemplary embodiment of a covering 40, in which a generally larger pore size has been set. It can be seen that some pores 41 have a size of more than 30 μm² wherein, however, a pore size of 300 μm² is not exceeded.

FIGS. 2 and 3 respectively show that the filaments 42 of the covering 40 intersect multiple times. A particular feature of the electrospinning process is, however, that in the covering 40, sites are present at which exclusively two filaments intersect, i.e. no more than two filaments 42 intersect. It is clear from this that the overall covering 40 has very thin walls and is therefore highly flexible.

The high flexibility of the covering 40 in combination with the high flexibility of the mesh structure 10 means that a stent 1 can be provided which can be introduced into a blood vessel through a very small balloon catheter 60. In particular, the balloon catheter 60 may have a size of 6 French, in particular at most 5 French, in particular at most 4 French, in particular at most 3 French, in particular at most 2 French. Specifically, in the exemplary embodiments described herein, the stents 1 in accordance with the exemplary embodiments described here may be combined with balloon catheters 60 which have an internal diameter of at most 991 μm (0.039 inches), in particular at most 686 μm (0.027 inches), in particular at most 635 μm (0.025 inches), in particular at most 533 μm (0.021 inches), in particular at most 432 μm (0.017 inches), in particular at most 406 μm (0.016 inches), in particular at most 381 μm (0.015 inches), in particular at most 330 μm (0.013 inches).

The layer thickness of the covering 40 in particularly preferred variations is at most 6 μm, in particular at most 4 μm, in particular at most 2 μm. In this, at most 4, in particular at most 3, in particular at most 2 filaments 42 intersect. In general, within the electrospun structure of the covering 40, points of intersection are present in which only 2 filaments 42 intersect.

Preferably, the mesh structure 10 has a cross sectional diameter in the neutral state of between 2.0 mm and 10 mm, in particular between 2.5 mm and 7 mm, in particular between 2.5 mm and 6 mm, in particular between 4.5 mm and 6 mm, in particular between 3.0 mm and 5 mm, in particular approximately 3.5 mm or approximately 4.5 mm. In general, the mesh structure 10 for the treatment of vulnerable plaques or soft plaques in intracranial blood vessels, for example the internal carotid artery (arteria carotis interna) or intracranial vessels distally therefrom, preferably has a cross sectional diameter of at most 6 mm, in particular between 2.5 mm and 5.5 mm. For the treatment of vulnerable plaques or soft plaques, preferably also for the treatment of stenoses, in extracranial blood vessels, in particular in extracranial sections of the carotid artery, for example the external carotid artery (arteria carotis externa), the mesh structure 10 may have a cross sectional diameter of at most 10 mm, in particular between at least 6 mm and at most 10 mm, in the neutral state.

FIG. 4 shows a braided mesh structure 10 which, in a preferred exemplary embodiment, can form a support for a covering 40. The braided mesh structure 10 is formed by a single wire 16 which is braided into a tube. The wire ends are connected within the mesh structure 10 with a connecting element 18.

The wire 16 has a plurality of sections which are designated as mesh elements 11, 12, 13, 14. Each section of the wire 16 which runs between two points of intersection 19 is described as an autonomous mesh element 11, 12, 13, 14. Clearly, four respective mesh elements 11, 12, 13, 14 delimit a mesh or cell 30.

The braided mesh structure 10 has flaring axial ends which are described as flares 17. The wire 16 is turned around in each flare 17 and forms end loops 15. Overall, in the exemplary embodiment shown, six end loops 15 are provided at each flare 17. Alternate end loops 15 carry an implant X-ray marker 50 in the form of a crimp sheath. Thus, three respective implant X-ray markers 50 are present on each axial end of the mesh structure 10.

FIGS. 5 and 6 show an exemplary embodiment of the stent 1 at different magnifications of a scanning electron microscopic image. The stent comprises a mesh structure 10 in accordance with FIG. 4 which is formed with a covering 40 produced from an electrospun fabric. The covering 40 is disposed on an outside of the tubular mesh structure 10.

FIG. 5 shows a 500-times magnification of a region of the device which comprises a cell tip 32 of the mesh structure 10. Two mesh elements or webs 11, 13 of a cell 30 meet in the cell tip 32. The covering 40 covers the webs 11, 12. It can be seen that the covering 40 has a plurality of different-sized pores 41, i.e. completely free through openings. The 3500-times magnification of FIG. 6 shows a section of the covering 40 of FIG. 5 in detail. The path of the individual filaments 42 of the electrospun fabric can clearly be seen. The filaments 42 delimit pores 41, wherein the pores 41 are irregular in configuration. However, it can be seen that some pores 41 have a larger open area than other pores 41. The larger pores 41 enable nutrients to pass through the covering 40.

FIG. 7 shows a balloon catheter 60 for delivering the stent 1 to a blood vessel. The balloon catheter 60 comprises two channels 61, 62. It is also possible to provide more than two channels 61, 62, for example three, four or more than four channels 61, 62.

The balloon catheter 60 furthermore comprises a balloon 63 which is disposed in the distal region of the channels 61, 62. As illustrated in FIG. 7, as well as in FIGS. 10 and 11, the balloon 63 is provided in the region of the catheter tip. The balloon 63 is at a distance from the outlet opening 64 of the delivery channel 62, so that a balloon-free section of the delivery channel 62 is formed between the outlet opening 64 and the distal balloon end 69. The balloon 63, in particular a proximal end 68 of the balloon 63, is in fluid communication with an inflation channel 61, as can be seen in FIGS. 10 and 11. The balloon 63 and the inflation channel 61 are aligned in the extended state of the balloon catheter 60. In order to connect the inflation channel 61 to the balloon 63, the wall of the inflation channel 61 is extended and transitions into the balloon wall. The transition between the inflation channel 61 and the balloon 63 is made by a continuous enlargement of the diameter between the inflation channel 61 and the maximum external circumference of the balloon 63 in the expanded state (see FIG. 10, FIG. 11).

The inflation channel 61 and the balloon 63 are formed as one piece. It is also possible to make the balloon 63 and the inflation channel 61 in two pieces and to provide an additional connecting piece between the balloon 63 and the inflation channel 61.

The connection in accordance with FIGS. 10 and 11 is particularly suitable for a coaxial arrangement of the two channels 61, 62 in accordance with FIG. 8. Here, the annular gap 72 between the two channels 61, 62 transitions into the internal volume of the balloon 63. The connection between the inflation channel 61 and the balloon 63 may be different, for example if the two channels 61, 62 are disposed adjacent to each other, as can be seen in FIG. 9. In this case, the connection between the balloon 63 and the inflation channel 61 is disposed laterally of the delivery channel 62 (not shown).

The inflation channel 61 serves to supply the balloon 63 with a fluid or for removal of the fluid from the balloon 63. As an example, the fluid may be a common salt solution or sterile water. The fluid may also be gaseous, for example ambient air. In practice, the fluid is frequently an air/liquid mixture.

The balloon catheter 60 comprises a delivery channel 62 with an outlet opening 64. The outlet opening 64 is disposed distally and connects the delivery channel 62 with the environment, in particular with the blood vessel, in which the stent 1 has been deployed. The outlet opening 64 as well as the internal diameter of the delivery channel 62 are adapted in a manner such that the delivery channel 62 has a retaining function in respect of the stent 1 disposed in the delivery channel 62. This means, for example, that the wall of the delivery channel 62 is strong enough to be able to accommodate the radial forces exerted by the self-expandable stent 1. Furthermore, the delivery channel 62 is sufficiently flexible for the catheter tip to be able to fit relatively narrow, contorted vessels.

As can be seen in FIGS. 7, 10 and 11, the delivery channel 62 extends through the balloon 63. This means that the balloon 63 is disposed around the outer circumference 71 of the delivery channel 62. The delivery channel 62 and the balloon 63 are disposed coaxially.

The association of the balloon 63 with the delivery channel 62, which is adapted for the delivery of the stent 1, has the advantage that the balloon catheter 60 has a dual function. Firstly, the balloon catheter 60 serves to deliver the stent 1 through the delivery channel 62. Secondly, by means of the balloon 63 disposed in the region of the catheter tip, the dilation or widening of the vessel which is required can be carried out without needing to change a catheter. In order to apply this principle, it may be sufficient for the delivery channel 62 to be generally associated with the balloon 63 and in fact in the region of the catheter tip, so that the balloon catheter 60 can be used both to deploy the stent 1 as well as for dilation, in particular pre-dilation of the stenosis and/or for post-widening of the implanted stent 1. The symmetrical arrangement of the delivery channel 62 and the balloon 63, as can be seen in FIGS. 7, 10 and 11, has the advantage that a simple radial widening of both the vessel as well as of the implanted stent 1 is possible, whereupon the delivery channel 62 is protected from the balloon 63 or, upon widening the balloon 63, it does not come into contact with the vessel or with the implanted stent 1.

The dual function of the balloon catheter 60 is achieved in that the delivery channel 62 is connected to a proximally disposed connector which is extracorporeal in use, which is adapted for introducing the stent 1 into the delivery channel 62. This means in practice that the extracorporeal connector is disposed at the proximal end of the catheter line 70, i.e. remote from the catheter tip. The extracorporeal connector for the delivery channel 62 is therefore directly accessible by the user. The connector may, for example, be adapted for loading the stent 1, wherein the stent 1 is moved from the extracorporeal connector up to the catheter tip through the delivery channel 62. As an alternative, the extracorporeal connector may be used in cooperation with a pre-loaded stent which is located in the region of the catheter tip, whereupon an actuating element can be moved through the extracorporeal connector and the delivery channel 62, for example a pusher or a guidewire with a slightly larger diameter than the stent 1, which is pushed ahead up to the pre-loaded stent 1 and then cooperates with it for deployment. The extracorporeal connector for the delivery channel 62 may comprise a loading lock for stents 1 which is known per se. The connector may be a Luer connector, for example.

As can be seen in FIG. 8, in one exemplary embodiment, the delivery channel 62 is coaxially disposed in the inflation channel 61. In this regard, an annular gap 72 is formed between the two channels 61, 62, which functions as a control lumen for the balloon 63. The delivery channel 62 which is disposed inside the inflation channel 61 forms the main lumen through which the stent 1 is moved.

FIG. 9 shows an alternative arrangement of the two channels 61, 62, wherein the working channel 61 and the delivery channel 62 are disposed adjacent to each other, in particular parallel to each other. Around the two channels 61, 62 is a catheter line 70 which fixes the arrangement of the two channels 61, 62. In general, the diameter of the delivery channel 62 is preferably larger than the diameter of the inflation channel 61.

FIGS. 10 and 11 show that the distal balloon end 69 is in fluid communication with the outer circumference 71 of the delivery channel 62. The proximal balloon end 68 is in fluid communication with the inflation channel 61. In this way, the balloon 63 has a tight fluid seal with the delivery channel 62, and on the other band can be inflated or deflate via the inflation channel 61.

The inflation channel 61 is connected to a proximally disposed connector which is extracorporeal in use. In the context of the extracorporeal connector for the delivery channel 62, a multiple connection, for example a Y Luer connector, is possible. The connector for the inflation channel 61 is either securely connected or releasably connected or connectable to a pressure device. The pressure device is configured to produce an over-pressure in order to inflate or to produce an under-pressure in order to deflate the balloon 63. The pressure device may, for example, comprise a syringe. Other pressure devices are possible.

The delivers/channel 62 is provided with a friction-reducing inner surface for movement of the stent 1 in translation in the delivery channel 62. Examples of a material for the inner surface are PTFE, FEP or HDPE or similar friction--reducing surface modifications. Other materials for the coating are also possible.

FIG. 11 shows that the catheter tip has a plurality of X-ray markers 65, 66, 67. A first X-ray marker 65 is disposed in the region of the distal outlet opening 64 and serves for the localisation of the end of the catheter tip. A second X-ray marker 66 is disposed in the region of the distal balloon end 69. A third X-ray marker 67 is disposed in the region of the proximal balloon end 68. The second and third X-ray markers 66, 67 act to establish the position of the balloon 63.

The catheter tip may be configured atraumatically and/or flexibly.

Suitable materials for the balloon catheter 60 are plastics, metals, shape memory materials such as nitinol, as well as radiopaque materials.

Furthermore, by means of the delivery channel 62, the balloon catheter 60 allows aspiration during or after dilation of the stenosis. In this regard, the delivery channel 62 is connected or connectable to a suction device. This has the advantage that particles of the vessel wall which become detached upon dilation can be sucked away through the delivery channel 62.

It is also possible to inject a contrast agent through the delivery channel 62. Specifically, after dilation of the stenosis, the delivery channel 62 of the balloon catheter 60 can be used to dispense the contrast agent in order to check whether the stenosis has been opened up. In this regard, the delivery channel 62 is connectable to or connected to an appropriate device for injection of a contrast agent, for example a syringe.

Furthermore, the balloon catheter 60 has the advantage that by means of a single balloon catheter 60, a plurality of stenoses can be dilated and/or a plurality of stents 1 can be deployed. A further advantage of the balloon catheter 60 is that upon dilation of the balloon 63, the delivery channel 62 does not collapse because it has an inherent stable channel wall.

The combination of the balloon catheter 60 described here with the stent 1 described here which has an electrospun covering 40 has been shown to be particularly advantageous in the treatment of stenoses. On the one hand, good pre-dilation of the stenosis is possible by means of the balloon catheter. On the other hand, good post-dilation can also be obtained. The stent 1 supports the dilated blood vessel well and in particular stabilizes vulnerable plaques because of its particularly flexible and dense covering 40. Furthermore, the stent 1 with its covering 40 permits good endothelial cell formation, which further stabilizes the dilated blood vessel.

### LIST OF REFERENCE INDICATORS

1 stent

10 mesh structure

11, 12, 13, 70 web or mesh element

15 end loop

16 wire

17 flare

18 connecting element

19 point of intersection

20 web connector

30 cell

31, 32 cell tip

34 cell ring

40 covering

41 pore

42 filament

50 implant X-ray marker

60 balloon catheter

61 inflation channel

62 delivery channel

63 balloon

64 distal outlet opening

65 first X-ray marker

66 second X-ray marker

67 third X-ray marker

68 proximal balloon end

69 distal balloon end

70 catheter line

71 outer circumference

72 annular gap

1-16. (canceled)

17. A medical system for the treatment of stenosis in intracranial blood vessels comprising:
a compressible and self-expandable implant for covering the stenosis, the implant having a mesh structure at least a section of which is provided with a covering produced from an electrospun fabric having irregularly sized pores; and
a balloon catheter for dilating the stenosis and for delivering the implant into the blood vessel.

18. The medical system as claimed in claim 17, wherein the covering comprises at least 10 pores with a size of between one of 5 μm² and 15 μm² or at least 15 μm² over an area of 100000 μm².

19. The medical system as claimed in claim 17, wherein the balloon catheter has at least two channels and a balloon, wherein an inflation channel of the at least two channels is in fluid communication with the balloon, and a delivery channel of the at least two channels extends through the balloon, and wherein the delivery channel has a proximal inlet opening and a distal outlet opening for deploying the implant.

20. The medical system as claimed in claim 19, wherein in a compressed state, the implant is one of configured to move through the delivery channel or disposed in the delivery channel.

21. The medical system as claimed in claim 19, wherein the balloon catheter comprises three X-ray markers, wherein a first X-ray marker is disposed in a region of the distal outlet opening of the delivery channel, a second X-ray marker is disposed in a region of a distal balloon end, and a third X-ray marker is disposed in a region of a proximal balloon end.

22. The medical system as claimed in claim 19, wherein the delivery channel has a friction-reducing inner coating for a translational movement of the implant in the delivery channel.

23. The medical system as claimed in claim 18, wherein each of the at least 10 pores of the covering has an inscribed diameter of at least 4 μm.

24. The medical system as claimed in claim 17, wherein the covering comprises at least 15 pores with a size of at least 30 μm²over an area of 100000 μm².

25. The medical system as claimed in claim 24, wherein the pores are at most 750 μm² in size.

26. The medical system as claimed in claim 17, wherein the covering is securely connected to the mesh structure.

27. The medical system as claimed in claim 17, wherein the mesh structure is sheathed with a bonding agent at least one of in parts or in sections, and wherein the bonding agent forms a mechanical interlock between the covering and the mesh structure.

28. The medical system as claimed in claim 17, wherein the covering is produced from a plastic material.

29. The medical system as claimed in claim 17, wherein the covering is formed from irregular filaments disposed in a network-like manner and having a filament thickness of between 0.1 μm and 3 μm.

30. The medical system as claimed in claim 17, wherein the covering has a biocompatible coating.

31. The medical system as claimed in claim 30, wherein the coating contains one of fibrin or heparin.

32. The medical system as claimed in claim 31, wherein the heparin is one of covalently bonded to the fibrin or incorporated into the fibrin.

33. A medical system for the treatment of stenosis in intracranial blood vessels comprising:
a compressible and self-expandable implant for covering the stenosis, the implant having a mesh structure at least a section of which is provided with a covering produced from an electrospun fabric having irregularly sized pores; and
a balloon catheter for dilating the stenosis and for delivering the implant into the blood vessel,
wherein the covering has at least 10 pores with a size of between one of 5 μm² and 15 μm² or at least 15 μm² over an area of 100000 μm².

34. The medical system as claimed in claim 33, wherein the balloon catheter has at least two channels and a balloon, wherein an inflation channel of the at least two channels is in fluid communication with the balloon, and a delivery channel of the at least two channels extends through the balloon, and wherein the delivery channel has a proximal inlet opening and a distal outlet opening for deploying the implant.

35. The medical system as claimed in claim 34, wherein the balloon catheter comprises three X-ray markers, wherein a first X-ray marker is disposed in a region of the distal outlet opening of the delivery channel, a second X-ray marker is disposed in a region of a distal balloon end, and a third X-ray marker is disposed in a region of a proximal balloon end.

36. A medical system for the treatment of stenosis in intracranial blood vessels comprising:
a compressible and self-expandable implant for covering the stenosis, the implant having a mesh structure at least a section of which is provided with a covering produced from an electrospun fabric having irregularly sized pores; and
a balloon catheter for dilating the stenosis and for delivering the implant into the blood vessel,
wherein the balloon catheter has at least an inflation channel, a delivery channel, and a balloon, wherein the inflation channel is in fluid communication with the balloon, and the delivery channel extends through the balloon, and wherein the delivery channel has a proximal inlet opening and a distal outlet opening for deploying the implant.
